(19) United States
(12) Patent Application Publication
(10) Pub. No.: US 2017/0049433 A1
(43) Pub. Date: Feb. 23, 2017

(54) HOLLOW SUTURE ANCHOR AND DRIVER

(71) Applicant: Smith & Nephew, Inc., Memphis, TN (US)

(72) Inventor: Michael Terry, Wilmette, IL (US)

(73) Assignee: Smith & Nephew, Inc., Memphis, TN (US)

(21) Appl. No.: 15/119,102

(22) PCT Filed: Feb. 19, 2015
(86) PCT No.: PCT/US15/16591

§ 371 Date: Aug. 15, 2016

Related U.S. Application Data
(60) Provisional application No. 61/942,459, filed on Feb. 20, 2014.

(51) Int. Cl. A61B 17/04

(52) U.S. Cl. CPC A61B 17/0401; A61B 2017/0409; A61B 2017/0412; A61B 2017/0414; A61B 2017/0427; A61B 2017/044; A61B 2017/0441; A61B 2017/0445

(57) ABSTRACT

Described herein are devices and techniques for no-hole-prep installation of miniaturized suture anchors into bone.

### DETAILED DESCRIPTION

The described technology relates generally to tissue repair, and more specifically, to an anchor for securing tissue to bone.

Arthroscopic surgery is a minimally invasive surgical procedure in which an examination and sometimes treatment of damage of the interior of a joint is performed using an arthroscope, a type of endoscope that is inserted into the joint through a small incision. Arthroscopic procedures, such as repairing a torn rotor cuff, often require soft tissue to be reattached to bone. To achieve this, anchors (sometimes called “suture anchors”) are placed in the bone and sutures attached to the anchor are passed through the tissue to securely retain the tissue in place. A procedure, and components for use in such procedure, that securely attaches tissue to bone using a plurality of attachment points over a large area of contact is needed. Such procedure must be able to be done in a quick and efficient manner with a minimum of recovery time for the patient. Such procedures make use of suture anchors to serve as attachment points for the tissue and sutures to the bone, generally requiring a surgeon to drill a bone-hole and then insert an anchor having sutures attached thereto or retained therein.

Traditional anchors are designed for sutures to be retained within the anchor and run through a cannulation in the shaft of the driver during insertion of the anchor into bone. Other anchors are designed for sutures to run through the anchor and be straddled by forked tongs extending from the end of the driver or a loop or suture secured to or in the anchor. However, to reduce the amount of bone stock removed by an anchor and minimize invasiveness, ever smaller anchors, sometimes constructed from a relatively brittle material composition (e.g., bioabsorbable or osteo-conductive materials), are being used. Miniaturized anchors result in reduced interior volume of the anchor, either precluding the use of cannulations and/or limiting miniaturization of the anchor. The miniaturization also results in weakened driver structures, often making the forked tongs too weak to withstand insertion and/or limiting miniaturization of the anchor. Additionally, the miniaturization results in weakened anchor structures. Due to these deficiencies, miniaturized anchors, if not properly supported, are susceptible to buckling, breaking, and/or failure during insertion into the bone. In some cases, these failures can cause complete structural failure of the anchor which may require removal of the anchor and/or damage to the insertion site while, in other cases, a partial failure or improper insertion can cause the suture to become disengaged from the anchor.

Due to the expense and time consumption associated with pre-drilling bone holes, there is a simultaneous desire to provide such small anchors with a capability of “no-hole-prep” insertion. No-hole-prep insertion refers to an anchor that can be inserted into the bone without pre-drilling. However, this configuration requires a significantly increased impact to penetrate the bone. This requirement only compounds the structural weakness problems described above, thereby further limiting the functionality and/or the achievable miniaturization of current suture anchors.

The foregoing needs are addressed by a hollow suture anchor and driver assembly. The hollow suture anchor includes a hollow interior, wherein the hollow interior has a variable cross-section for mating with a shaft of the driver and a bore transverse to the hollow interior for receiving a suture. The shaft of the driver has a variable cross-section designed to displace the suture in the bore and mate with the variable cross-section of the hollow interior to form a suture passage between the anchor and the shaft. The shaft of the driver, when mated to the hollow interior, is also configured to support the hollow interior of anchor. The driver also includes a distal tip designed to protrude from a distal end of the anchor when the anchor and the driver are mated.

In one aspect, the present disclosure relates to a system for tissue repair. The system includes a suture anchor having a longitudinal axis. The suture anchor includes a body defining a hollow interior, the hollow interior including a proximal region having a first cross-section, a distal region having a second cross-section, and a medial transition region positioned between the proximal region and the distal region. The suture anchor also includes diametrically opposed first and second apertures in the body, forming a bore extending through the body transversely to the longitudinal axis, the bore sized to receive one or more sutures. The system for tissue repair also includes a driver. The driver includes a shaft. The shaft includes a proximal portion having a first complementary cross-section, wherein the first complementary cross-section is an inverse shape complementary to the first cross-section, the proximal portion adapted to engage with the proximal region of the suture anchor. The shaft also includes a distal portion having a second complementary cross-section, wherein the second complementary cross-section is an inverse shape complementary to the second cross-section, the distal portion adapted to engage with the distal region of the suture anchor. The shaft also includes a medial transition portion configured to mate with the medial transition region of the body to form a suture passage, the suture passage in communication with the bore and adapted for routing the one or more sutures around the shaft. The shaft also includes a tip extending distally from the distal portion of the shaft.

Any of the aspects and/or embodiments described herein can include one or more of the following embodiments. In some embodiments, the body includes at least one open helical coil, wherein the hollow interior is in communication with a region exterior to the at least one open helical coil through a spacing between turns of the at least one open helical coil. In some embodiments, the diametrically opposed first and second apertures of the bore are coincident with the spacing between turns of the at least one open helical coil. In some embodiments, the system for tissue repair includes at least one drive surface connected to at least two turns of the at least one open helical coil.

In some embodiments, the body includes a sleeve, wherein an internal surface of the sleeve defines the hollow interior. In some embodiments, the body includes one or more protrusions extending from an external surface of the sleeve. In some embodiments, the one or more protrusions include one or more screw threads and/or helical coils defined along at least a portion of the external surface of the sleeve. In some embodiments, the one or more protrusions include a plurality of stacked ribs defined around at least a portion of the external surface of the sleeve. In some embodiments, the diametrically opposed first and second apertures of the bore are formed in the sleeve. In some embodiments, they system for tissue repair includes one or more channels defined along at least a portion of an external surface of the suture anchor and extending along the longitudinal axis proximally from the bore. In some embodiments, the tip is a bone insertion tip.

In one aspect, the present disclosure relates to a method for tissue repair. The method includes providing an anchor having a bore and one or more sutures installed in the bore, the bore being transverse to a longitudinal axis of the anchor and extending through a hollow body of the anchor, the hollow body having a proximal region having a first cross-section, a distal region having a second cross-section, and a medial transition region positioned between the proximal region and the distal region. The method also includes inserting a shaft of a driver into the hollow body of the anchor, the shaft having a proximal portion having a first complementary cross-section, wherein the first complementary cross-section is an inverse shape complementary to the first cross-section, the proximal portion adapted to engage with the proximal region of the hollow body, a distal portion having a second complementary cross-section, wherein the second complementary cross-section is an inverse shape complementary to the second cross-section, the distal portion adapted to engage with the distal region of the hollow body, and a medial transition portion configured to mate with the medial transition region of the hollow body to form a suture passage, the suture passage in communication with the bore and adapted for routing the one or more sutures around the shaft, and, the shaft including a tip extending distally from the distal portion of the shaft. The method also includes routing one or more sutures around the shaft through the suture passage. The method also includes installing the anchor into a bone. The method also includes tensioning the one or more sutures.

Any of the aspects and/or embodiments described herein can include one or more of the following embodiments. In some embodiments, the method includes threading one or more sutures through the bore of the anchor. In some embodiments, installing the anchor into the bone includes positioning the tip against the bone. In some embodiments, installing the anchor into the bone includes applying an insertion force to the driver, wherein applying the insertion force causes the tip to penetrate a surface of the bone. In some embodiments, installing the anchor into the bone includes continuing to apply the insertion force to the driver until the anchor is fully inserted into the bone. In some embodiments, installing the anchor into the bone includes terminating application of the insertion force when the distal region of the anchor contacts the surface of the bone. In some embodiments, installing the anchor into the bone includes screwing the anchor into the bone by twisting the driver until the anchor is fully inserted into the bone.

The methods and systems for a hollow suture anchor and driver can provide one or more of the following advantages. One advantage of the technology is that the suture anchor is supported by the driver shaft which creates a more robust construct for insertion of the anchor. Another advantage of the technology is that the anchor tip of the driver receives a portion of the insertion force, thereby protecting the suture anchor. Still another advantage of the technology is that the suture passage is defined by the driver shaft and the anchor, thereby minimizing structural weakening of the anchor and the driver.

The present disclosure is further described in the following detailed description, in reference to the noted plurality of drawings by way of non-limiting examples of embodiments of the present disclosure, in which like reference numerals represent similar parts throughout the several views of the drawings.

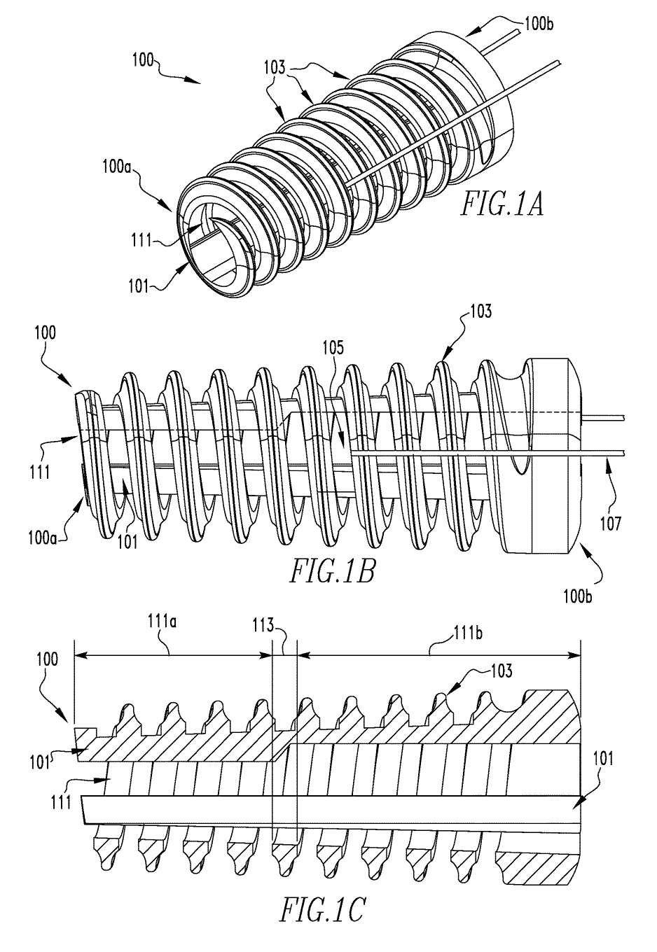

FIGS. 1A-1B are side views illustrating an example hollow suture anchor in accordance with various embodiments.

FIG. 1C is a cross-sectional view illustrating an example hollow suture anchor in accordance with various embodiments.

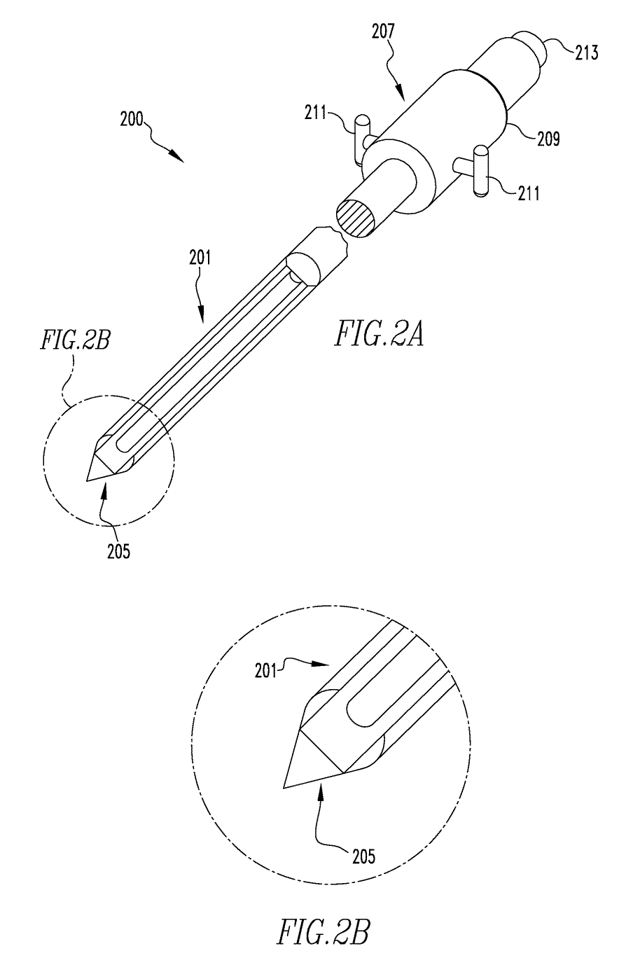

FIGS. 2A-2B are isometric views illustrating various components of an driver in accordance with various embodiments.

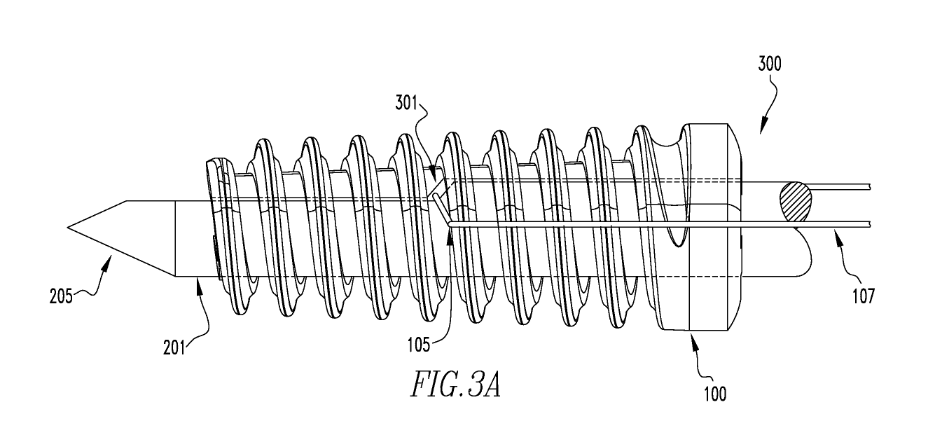

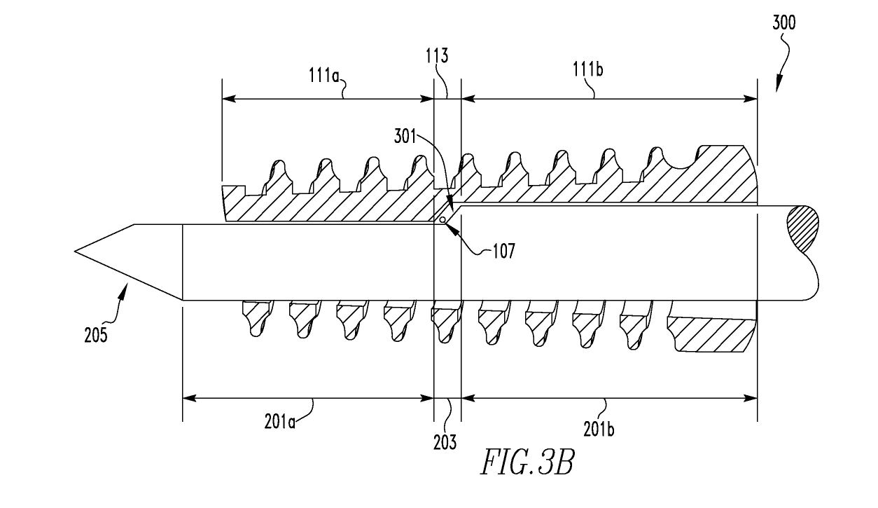

FIG. 3A is a side view illustrating a hollow suture anchor and driver assembly in accordance with various embodiments.

FIG. 3B is a cross-sectional view illustrating a hollow suture anchor and driver assembly in accordance with various embodiments.

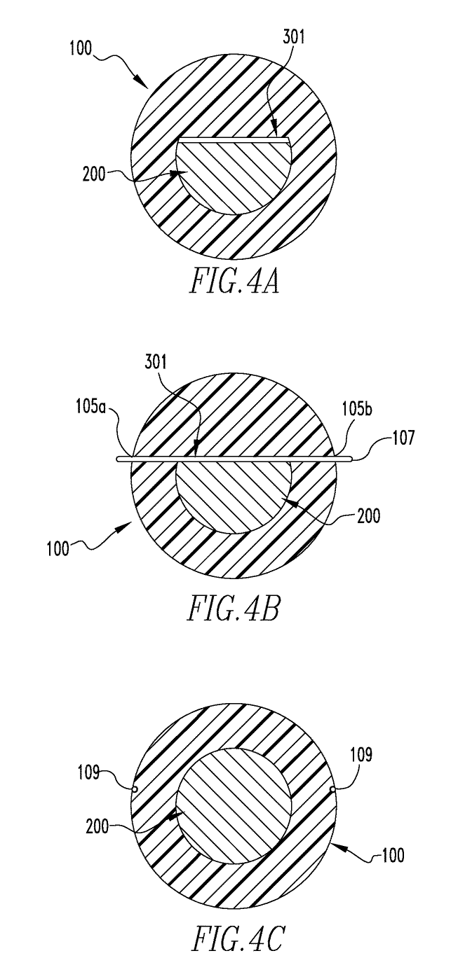

FIGS. 4A-4C are cross-sectional top views illustrating distal, medial, and proximal portions of a hollow suture anchor and driver assembly in accordance with various embodiments.

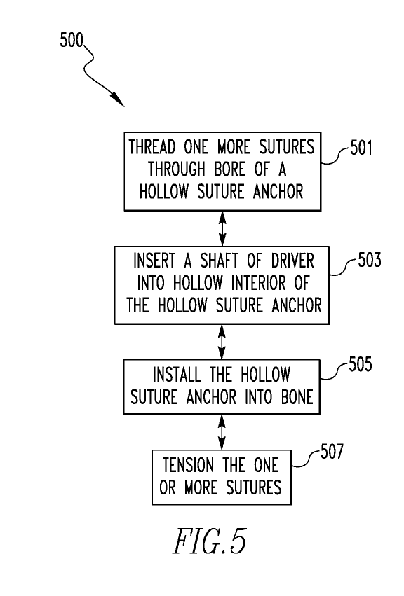

FIG. 5 is a flow chart illustrating a method for using a hollow suture anchor and driver assembly in accordance with various embodiments.

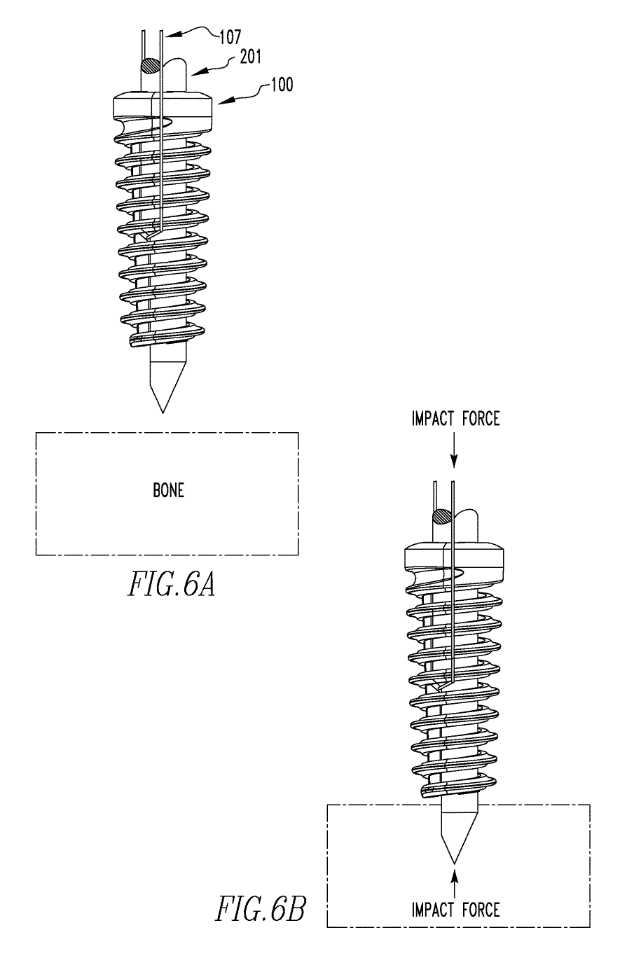

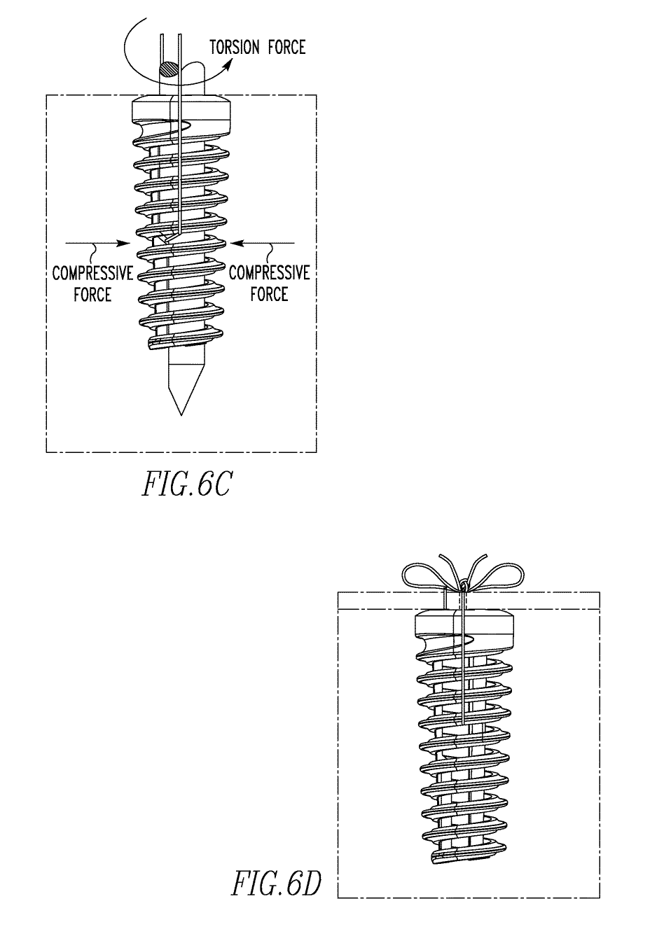

FIGS. 6A-6D are a series of side views illustrating various stages of installing a hollow suture anchor into bone using a hollow suture anchor and driver assembly.

In the following detailed description of the illustrated embodiments, reference is made to accompanying drawings, which form a part thereof, and within which are shown by way of illustration, specific embodiments, by which the subject matter can be practiced. It is to be understood that other embodiments can be utilized and structural changes can be made without departing from the scope of the disclosure.

The particulars shown herein are by way of example and for purposes of illustrative discussion of the embodiments only and are presented in the case of providing what is believed to be the most useful and readily understood description of the principles and conceptual aspects of the disclosure. In this regard, no attempt is made to show structural details of the subject matter in more detail than is necessary for the fundamental understanding of the disclosure, the description taken with the drawings making apparent to those skilled in that how the several forms of the present disclosure can be embodied in practice. Further, like reference numbers and designations in the various drawings indicate like elements.

A hollow suture anchor and driver assembly as provided herein can be used by a surgeon to install the hollow suture anchor into a bone during a surgical procedure. The installation can, in accordance with various embodiments, proceed as shown in FIGS. 6A-6D. As shown in FIG. 6A, the anchor can be assembled on the driver such that one or more sutures have been passed through a transverse bore of the anchor. It will be apparent in view of this disclosure that transverse bore, as used herein, can refer to a through-hole made through an outer surface of a closed-architecture anchor or can refer to a gap between ribs and/or screw threads of an open-architecture anchor (e.g., as shown in FIGS. 6A-6D). As shown in FIG. 6B, the tip of the driver can, in accordance with various embodiments, be pounded into the bone by the surgeon, thereby imparting various forces onto the assembly. These forces, as shown in FIG. 6B, can include, for example, impact forces from the pounding. As shown in FIG. 6C, the anchor can then be rotated (screwed) into the bone until the anchor is fully inserted into the bone, thereby imparting various other forces onto the assembly. These forces, as shown in FIG. 6C, can include, for example, torsion forces from the rotation of the driver and/or compressive forces from the surrounding bone. Additional forces that can be exerted on the anchor during installation of the anchor include, for example, bending and/or buckling forces. Such forces are generally introduced if the driver angle is changed during insertion or just prior to insertion.

It will be apparent in view of the present disclosure that other anchor configurations and insertion techniques (e.g., anchors having external ribs rather than screw threads, which are pounded into the bone until full insertion is achieved) are contemplated.

Following full insertion of the anchor, as shown in FIG. 6D, the driver is removed, the sutures are attached to a tissue (e.g., a rotator cuff, tissue graft, and/or other bodily tissue), the tissue is drawn into a desired position by tensioning the sutures, and the tissue is then fixated to the bone by securing the sutures in place (e.g., by tying a knot in the suture). It will be apparent in view of the present disclosure that the sutures, in accordance with various embodiments, can be attached to the tissue before insertion of the anchor, at any stage of the insertion of the anchor, after full insertion of the anchor but before removal of the driver, and/or after full insertion of the anchor and removal of the driver. It will be further apparent in view of this disclosure that the tensioning can at least partially occur at any time after the sutures are attached to the tissue in accordance with various embodiments.

It will be apparent in view of this disclosure that the installation described above can be used for any portion of any suitable surgical procedure. For example and without limitation, the anchor of the hollow suture anchor and driver assembly can be used in a double row footprint repair, where the suture is taken from another anchor that was previously inserted into bone. Also for example and without limitation, the anchor of the hollow suture anchor and driver assembly can be used as a medial row anchor in a double row repair and the suture can be placed in another anchor that is subsequently inserted into bone, rather than being tied into a knot.

As shown in FIGS. 1A-4C, a hollow suture anchor and driver assembly 300 is provided herein that includes a hollow suture anchor 100 and a driver 200. As best shown in FIGS. 1C and 3A-3B, the hollow suture anchor 100 includes a hollow interior 111, wherein the hollow interior 111 has a variable cross-section for mating with a shaft 201 of a driver 200 and a bore 105 defined by diametrically opposed apertures in the hollow suture anchor (e.g., the apertures are coincident with the spacing between turns of the open helical coil of the anchor shown in FIGS. 1A-1C) transverse to the hollow interior 111 for receiving a flexible member 107. The shaft 201 of the driver 200 has a variable cross-section designed to displace the flexible member 107 in the bore 105 and mate with the variable cross-section of the hollow interior 111 to form a suture passage 301 for routing the suture around the shaft 201 between the anchor 100 and the driver 200. By routing the flexible members 107 around the shaft 201 between the hollow suture anchor 100 and the driver 200, the space limitations on internal driver cannulations and the structural limitations on forked tongs are both avoided.

As shown in FIG. 3B, the shaft 201 of the driver 200, when mated to the hollow interior 111, is also configured to be in contact with an internal surface of the hollow interior 111 of the anchor 100 along substantially its entire longitudinal length. By maintaining contact between the internal surface of the hollow interior 111 and the shaft 201, the system 300 advantageously provides for structural reinforcement of the anchor 100 by the driver 200, thereby increasing the structural integrity of the anchor 100 and helping to resist the forces exerted on the anchor 100 during installation (e.g., the compressive forces as shown in FIG. 6C). Even if the shaft 201 does not contact with the internal surface of the hollow interior 111 at the suture passage 301, the flexible members 107 within the passage 301 help to provide reinforcement for the anchor 100.

As best illustrated by FIGS. 2B and 3A-3B, the driver 200 also includes a distal tip 205 designed to protrude from a distal end 101a of the anchor 100 when the anchor 100 and the driver 200 are mated. The distal tip 205 of the driver 200 is thereby positioned to lead the anchor 100 into bone and will receive a portion of the impact forces required for an installation, thereby protecting the anchor 100 from installation forces and making no-hole-prep installation of miniaturized anchors feasible.

The hollow suture anchor and driver assembly 300 includes the hollow suture anchor 100 having a central longitudinal axis. Referring now to FIGS. 1A-1C, the hollow suture anchor 100 includes a distal end 100a and a proximal end 100b and one or more drive surfaces 101. In various embodiments, the hollow suture anchor 100 includes one or more threads 103 connected by the drive surfaces 101 for fixing the hollow suture anchor 100 in the bone. The threads 103 define the hollow interior 111 for receiving the driver 200 (shown in FIGS. 2A-3D), the hollow interior 111 having the variable cross-section between the proximal region 111b and the distal region 111a. The hollow interior 111 is open at the distal end 100a and at the proximal end 100b. The hollow suture anchor 100 also includes the bore 105 transverse to the longitudinal axis of the hollow suture anchor 100 and sized to receive one or more flexible members 107. In various embodiments, the hollow suture anchor optionally includes one or more channels 109, extending along the longitudinal axis from the bore 105 toward the proximal end 100b. In accordance with various embodiments, the channels 109 are configured for at least partially holding the one or more flexible members 107.

The anchor 100, in accordance with various embodiments can be made from a non-metal material, such as a polymer material (e.g., PEEK, nylon, polyester, PVDF, and/or polypropylene) and/or absorbable materials (e.g., polyglycolic acid, polylactic acid, monocryl, and/or polydioxanone), but can also be made from a metal material (e.g., surgical steel or titanium). The non-metal material may include growth factors that would allow for faster healing. Absorbable materials are designed for slow absorption by the body. Generally, such absorbable materials are designed with an absorption rate configured to prevent the anchor 100 and the flexible members 107 from being absorbed by the body until the soft tissue begins to grow into the bone and become re-attached to it.

Flexible members 107 can include any suitable member including, for example, wire, sutures and/or suture tape. Flexible members 107 can be made from any suitable material, including for example, catgut, silk, absorbable materials, polymer material, metals, and/or any other suitable material and may include growth factors that would allow for faster healing.

As shown in FIG. 1C and FIGS. 3A-4C, the cross-sectional area and/or shape of the hollow interior 111 changes, at medial transition area 113, from a cross-section of the proximal region 111b to a cross-section of the distal region 111a. In accordance with various embodiments, at the medial transition region 113, a transitional surface (e.g., a filleted, curved, sloped, and/or stepped surface) begins to incur into the hollow interior 111, thereby transitioning between a larger cross-sectional area of the proximal region 111b and a smaller cross-sectional area of the distal region 111a. In accordance with various embodiments, the proximal region 111b and the distal region 111a of the hollow interior 111 are configured to engage a proximal portion 201b and a distal portion 201a of the shaft 201 as shown in FIGS. 2A-4C. In accordance with various embodiments, the anchor 100 and driver 200 are configured to support the interior surface of the proximal region 111b by engagement (e.g., frictional surface contact) with the proximal portion 201b and to support the interior surface of the distal region 111a by engagement with the distal portion 201a. In accordance with various embodiments, a cross-section of the proximal portion 201b is a complementary inverse shape with the cross-section of the proximal region 111b and a cross-section of the distal portion 201a is a complementary inverse shape with the cross-section of the distal region 111a. This arrangement results in an advantage because the driver 200 supports the anchor 100, thereby absorbing a portion of the insertion forces exerted on the anchor 100 (e.g., compressive forces from the bone, torsion forces from the driver, bending forces, and/or buckling forces as described above).

In accordance with various embodiments, the medial transition region 113 of the hollow suture anchor 100 is configured to mate with a medial transition portion 203 of the driver 200 at a longitudinal position coincident with the bore 105, thereby defining the suture passage 301 around the shaft 201 between the medial transition region 113 and the medial transition portion 203. As shown in FIG. 3B, at the medial transition portion 203, a transitional surface (e.g., a filleted, curved, sloped, and/or stepped surface) begins to recede from the shaft 201, thereby transitioning between a larger cross-sectional area of the proximal portion 201b and a smaller cross-sectional area of the distal portion 201a. As shown in FIGS. 3B and 4B, the suture passage 301 begins at a first opening 105a of the bore 105, extends around the shaft 201 within the hollow interior 111, and ends at a second opening 105b of the bore 105. Forming the suture passage 301 in this manner results in an advantage because a minimum of material is removed from each of the anchor 100 and the driver 200 to accommodate the flexible member(s) 107. Guiding the suture(s) around the driver 200 provides further advantage by eliminating the need for forked tongs and allowing the driver 200 to have a solid, stronger structure.

In accordance with various embodiments, one or more flexible members 107 are passed through the first opening 105a and the second opening 105b of the bore 105 prior to mating with the driver 200. When mated with the suture anchor, the distal tip 205, the distal portion 201a, and the proximal portion 201b of the driver 200 are inserted into the hollow interior 111. After insertion of the driver 200, the flexible member(s) 107 are displaced by the shaft 201 and thereby routed around the shaft 201 through the suture passage 301 defined between the medial transition region 113 of the hollow interior 111 and the medial transition portion 203 of the shaft 201. In accordance with various embodiments, the one or more flexible members 107 are passed through the first opening 105a of the bore 105, around the shaft 201 through the suture passage 301, and through the second opening 105b of the bore 105 after the anchor 100 is mated with the driver 200. In accordance with various embodiments, the system 300 can be provided as an assembly with the one or more flexible members 107 pre-installed in the suture passage 301 and the bore 105. Passing the suture can be performed by any known technique (e.g., using a suture passer, threading a free end of each flexible member 107 through the bore 105, and/or any other suitable technique).

In accordance with various embodiments, one and/or both of the transition regions 113, 203 can be eliminated by defining a channel solely along an exterior circumference of the shaft 201 and/or by defining a channel solely along an interior circumference of the hollow interior 111.

In accordance with various embodiments, each end of the flexible member(s) 107 is run through channel(s) 109 as best shown in FIG. 4C. After the anchor has been inserted into bone, the flexible member(s) 107 are slidable in the channel(s) 109 and the bore 105 during the sliding of a knot into place and/or in order to adjust a tension of the flexible member(s) 107 prior to fixing them in place with a knot or other fixing means. Providing channels 109 to allow slidability of the flexible member(s) 107 after installation of the anchor 100 into bone advantageously permits the surgeon to be more precise in achieving the desired tension. In various embodiments, the one or more channels 109 are defined in an exterior surface of the threads 103 and extend from the bore 105 toward the proximal end 100b. In accordance with various embodiments, the channels 109 can be defined in the threads 103, an exterior surface of the drive surfaces 101, or both. The one or more channels 109, in accordance with various embodiments, are sized to at least partially hold the one or more flexible members 107.

In accordance with various embodiments, the one or more threads 103 can include screw threads and/or helical threads extending around the hollow interior 111 along at least a portion of a longitudinal length of the drive surfaces 101.

As shown in FIGS. 2A-2B, the driver 200 includes the shaft 201 having the distal portion 201a, the proximal portion 201b, and the medial transition portion 203 as described above. The driver also includes the distal tip 205 extending from the distal portion 201a and a handle 207 attached to the shaft 201 opposite the distal tip 205.

The distal tip 205, in accordance with various embodiments, is configured to pass through the hollow suture anchor 100 and, when the driver 200 is mated to the hollow suture anchor 100, extends distal from the distal end 100a of the hollow suture anchor 100. In accordance with various embodiments, the distal tip 205 can be configured to penetrate a surface of the bone and provide a tapered lead-in for the hollow suture anchor 100 during insertion. In accordance with various embodiments, the distal tip 205 can be advantageously configured to absorb at least a portion of the insertion forces, thereby at least partially protecting the anchor 100 from the insertion forces. In such embodiments, the distal tip 205 can advantageously be constructed to withstand insertion forces associated with a no-hole-prep insertion (e.g., pound-in impact forces of sufficient strength to cause the distal tip 205 to penetrate the bone). Although the distal tip 205 is depicted herein as a sharp, pointed tip, it will be apparent in view of this disclosure that any shape and/or design suitable for creation of a hole in bone can be used in accordance with various embodiments.

As shown in FIG. 2A, the driver also includes a handle 207 attached to the shaft 201 opposite the distal tip 205. The handle 207 can, in accordance with various embodiments, include a grip section 209 for being held by a surgeon. In accordance with various embodiments, the handle can include one or more suture holders 211 for releasably retaining one or more flexible members 107 in place during installation of the hollow suture anchor 100 into bone. The handle, in accordance with various embodiments, can also include a pounding surface 213 for receiving an insertion force to be transmitted to the shaft 201 and the distal tip 205.

Referring now to FIG. 5, a method 500 of tissue repair, in accordance with various embodiments, includes the steps of threading 501 one or more sutures through a bore of a hollow suture anchor, inserting 503 a shaft of a driver into a hollow interior of the hollow suture anchor, installing 505 the hollow suture anchor into bone, and tensioning 507 the one or more sutures.

The step of threading 501, in accordance with various embodiments, can include, for example but not limited to threading one or more flexible members 107 through the bore 105 of the hollow suture anchor 100 and/or the suture passage 301 formed between the anchor 100 and the driver 200 as described above with reference to FIGS. 1A-4C.

The step of inserting 503, in accordance with various embodiments, can include, for example but not limited to, passing the distal tip 205 through the hollow suture anchor 100, supporting the interior surface of the proximal region 111b by engagement with the proximal portion 201b, and supporting the interior surface of the distal region 111a by engagement with the proximal portion 201a as described above with reference to FIGS. 1A-4C into the hollow interior 111, thereby mating the anchor 100 and driver 200.

In accordance with various embodiments, where the one or more flexible members 107 are threaded through the bore 105 prior to mating of the anchor 100 with the driver 200, the flexible members 107 are displaced by the shaft 201 during insertion and are retained in the suture passage 301 defined between the medial transition region 113 of the hollow interior 111 and the medial transition portion 203 of the shaft 201 as described above with reference to FIGS. 1A-4C.

The step of installing 505, in accordance with various embodiments, can include, for example but not limited to, pound-in (e.g., as shown in FIGS. 5A-5D), screw-in, pre-drilled, and/or no-hole-prep (e.g., as shown in FIGS. 5A-5D) installation.

In accordance with various embodiments, as shown, for example but not limited to, in FIGS. 6A-6D, a surgeon can pound the pounding surface 213 of the driver 200 (e.g., using a mallet or hammer) to transmit an installation force to the handle 207. The force is then transmitted from the handle 207 to the shaft 201 and from the shaft 201 to the distal tip 205 as described above with reference to FIGS. 2A-4C. In accordance with various embodiments, the transmitted force can be sufficient to drive the distal tip 205 into a pre-drilled bone hole. In accordance with various embodiments, the transmitted force can be sufficient to drive the tip into an unprepared bone surface.

As shown in FIGS. 6A-6D and in accordance with various embodiments, the surgeon stops pounding after the distal end 100a of the hollow suture anchor 100 and the lead ends of the threads 103 are brought into contact with the bone. The surgeon can then twist the grip 209 to advance the hollow suture anchor 100 into the bone.

The step of tensioning 507, in accordance with various embodiments, can include, for example but not limited to, sliding the one or more flexible members 107 in the channels 109 and the bore 105 until a desired tension is achieved as described above with reference to FIGS. 1A-4C. In accordance with various embodiments, once a desired tension is achieved, one or more free ends of the flexible members 107 can be placed into the one or more suture holders 211 to temporarily retain the flexible members 107 in a tensioned state until a more permanent means of fixation can be achieved (e.g., tying a knot to fix the suture in place).

While shown and described above as an open-architecture hollow suture anchor, it will be apparent in view of this disclosure that closed-architecture hollow suture anchors such as, for example, an anchor having a sleeve-type body with or without protrusions extending therefrom or a threaded anchor having webbing disposed between turns of the threads can also be used in accordance with various embodiments. In accordance with various such embodiments, the hollow suture anchor and driver assembly includes the hollow suture anchor having a central longitudinal axis. The hollow suture anchor includes a sleeve-type body having a distal end and a proximal end. The body includes the hollow interior for receiving the driver, the hollow interior having the variable cross-section between the proximal region and the distal region. The hollow interior of the body is open at the distal end and at the proximal end. The hollow suture anchor also includes the bore transverse to the longitudinal axis of the hollow suture anchor and sized to receive one or more sutures. In such embodiments the bore can be defined by diametrically opposed apertures in the sleeve and/or the webbing between turns of the threads.

In such embodiments, the hollow suture anchor optionally includes one or more channels, extending along the longitudinal axis from the bore toward the proximal end. In accordance with various embodiments, the channels are configured for at least partially holding the one or more sutures. In various embodiments, the hollow suture anchor optionally includes one or more protrusions projecting from an exterior surface of the body for fixing the hollow suture anchor in the bone. In accordance with various embodiments, the channels are defined in an external surface of the sleeve, in the protrusions, or both

In accordance with various embodiments, the one or more protrusions projecting from an exterior surface of the body can include circumferential rings encircling the body and stacked along at least a portion of a longitudinal length of the body. In accordance with various embodiments, the one or more protrusions projecting from an exterior surface of the body can include screw threads and/or helical threads extending around the body along at least a portion of a longitudinal length of the body. In accordance with various embodiments, the one or more protrusions projecting from an exterior surface of the body can include a plurality of barbs protruding from the body.

The driver includes the shaft having the distal portion, the proximal portion, and the medial transition portion as described above. The driver also includes the tip extending from the distal portion and a handle attached to the shaft opposite the tip.

The tip, in accordance with various embodiments, is configured to pass through the hollow suture anchor and, when the driver is mated to the hollow suture anchor, extends distal from the distal end of the hollow suture anchor. In accordance with various embodiments, the tip can be configured to penetrate a surface of the bone and provide a tapered lead-in for the hollow suture anchor during insertion. In accordance with various embodiments, the tip can be advantageously configured to absorb at least a portion of the insertion forces, thereby at least partially protecting the anchor from the insertion forces. In such embodiments, the tip can advantageously be constructed to withstand insertion forces associated with a no-hole-prep insertion (e.g., pound-in impact forces of sufficient strength to cause the distal tip to penetrate the bone).

The driver also includes a handle attached to the shaft opposite the tip. The handle can, in accordance with various embodiments, include a grip section for being held by a surgeon. In accordance with various embodiments, the handle can include one or more suture holders for releasably retaining one or more sutures in place during installation of the hollow suture anchor into bone. The handle, in accordance with various embodiments, can also include a pounding surface for receiving an insertion force to be transmitted to the shaft and the tip.

A method of tissue repair using a closed-architecture anchor, in accordance with various embodiments, includes the steps of threading one or more sutures through a bore of a hollow suture anchor, inserting a shaft of a driver into a hollow interior of the hollow suture anchor, installing the hollow suture anchor into bone, and tensioning the one or more sutures.

The step of threading, in accordance with various embodiments, can include, for example but not limited to threading one or more sutures through the bore of the hollow suture anchor and/or the suture passage formed between the anchor and the driver as described above.

The step of inserting, in accordance with various embodiments, can include, for example but not limited to, passing the distal tip through the hollow suture anchor, supporting the interior surface of the proximal region by engagement with the proximal portion, and supporting the interior surface of the distal region by engagement with the proximal portion as described above into the hollow interior, thereby mating the anchor and driver.

In accordance with various embodiments, where the one or more sutures are threaded through the bore prior to mating of the anchor with the driver, the sutures are displaced by the shaft during insertion and are retained in the suture passage defined between the medial transition region of the hollow interior and the medial transition portion of the shaft as described above.

The step of installing, in accordance with various embodiments, can include, for example but not limited to, pound-in, screw-in, pre-drilled, and/or no-hole-prep installation.

In accordance with various embodiments, a surgeon can pound the pounding surface of the driver (e.g., using a mallet or hammer) to transmit an installation force to the handle. The force is then transmitted from the handle to the shaft and from the shaft to the distal tip as described above. In accordance with various embodiments, the transmitted force can be sufficient to drive the distal tip into a pre-drilled bone hole. In accordance with various embodiments, the transmitted force can be sufficient to drive the tip into an unprepared bone surface.

In accordance with some embodiments, the surgeon can continue pounding until the hollow suture anchor has been driven to full insertion depth. In some embodiments, the protrusions (e.g., ribs or barbs) of the hollow suture anchor can be configured to aid retention of the anchor in the bone after the anchor is driven to full insertion depth.

In accordance with some embodiments, the surgeon can stop pounding after the distal end of the hollow suture anchor and the lead ends of the protrusions (e.g., screw threads or helical threads) are brought into contact with the bone. The surgeon can then twist the grip to advance the hollow suture anchor into the bone.

The step of tensioning, in accordance with various embodiments, can include, for example but not limited to, sliding the one or more sutures in the channels and the bore until a desired tension is achieved as described above. In accordance with various embodiments, once a desired tension is achieved, one or more free ends of the sutures can be placed into the one or more suture holders to temporarily retain the sutures in a tensioned state until a more permanent means of fixation can be achieved (e.g., tying a knot to fix the suture in place).

Although the present disclosure has been described herein with reference to particular means, materials and embodiments, the present disclosure is not intended to be limited to the particulars disclosed herein; rather, the present disclosure extends to all functionally equivalent structures, methods and uses, such as are within the scope of the appended claims.

What is claimed is:

1. A system for tissue repair comprising:
a suture anchor having a longitudinal axis and including:
a body defining a hollow interior, the hollow interior including a proximal region having a first cross-section, a distal region having a second cross-section, and a medial transition region positioned between the proximal region and the distal region, and;
diametrically opposed first and second apertures in the body, forming a bore extending through the body transversely to the longitudinal axis, the bore sized to receive one or more flexible members; and
a driver including:
a shaft having:
a proximal portion having a first complementary cross-section, wherein the first complementary cross-section is an inverse shape complementary to the first cross-section, the proximal portion adapted to engage with the proximal region of the body of the suture anchor;
a distal portion having a second complementary cross-section, wherein the second complementary cross-section is an inverse shape complementary to the second cross-section, the distal portion adapted to engage with the distal region of the body of the suture anchor;
a medial transition portion extending between the proximal portion and the distal portion, the medial transition portion configured to mate with the medial transition region of the body of the suture anchor to form a suture passage, the suture passage in communication with the bore and adapted for routing the one or more flexible members around the shaft, and;
a tip extending distally from the distal portion of the shaft and beyond the distal region of the body of the suture anchor.

2. The system for tissue repair of claim 1, wherein the body is an open helical coil having at least one drive surface connected to at least two turns of the open helical coil, wherein the hollow interior is in communication with a region exterior to the open helical coil through a spacing between turns of the open helical coil.

3. The system for tissue repair of claim 2, wherein the diametrically opposed first and second apertures of the bore are coincident with the spacing between turns of the open helical coil.

4. The system for tissue repair of claim 1, wherein the body includes one or more protrusions extending from an external surface of the body.

5. The system for tissue repair of claim 4, wherein the one or more protrusions include a screw thread defined along at least a portion of the external surface of the body.

6. The system for tissue repair of claim 4, wherein the one or more protrusions include a plurality of stacked ribs defined around at least a portion of the external surface of the body.

7. The system for tissue repair of claim 4, wherein the diametrically opposed first and second apertures of the bore are formed in the external surface of the body.

8. The system for tissue repair of claim 1, further comprising one or more channels defined along at least a portion of an external surface of the body of the suture anchor and extending along the longitudinal axis proximally from the bore.

9. The system for tissue repair of claim 1, wherein the tip is a bone insertion tip.

10. A method for tissue repair comprising:
providing an anchor having a bore and one or more flexible members installed in the bore, the bore being transverse to a longitudinal axis of the anchor and extending through a hollow body of the anchor, the hollow body having a proximal region having a first cross-section, a distal region having a second cross-section, and a medial transition region positioned between the proximal region and the distal region;
inserting a shaft of a driver into the hollow body of the anchor, the shaft having a proximal portion having a first complementary cross-section, wherein the first complementary cross-section is an inverse shape complementary to the first cross-section, the proximal portion adapted to engage with the proximal region of the hollow body, a distal portion having a second complementary cross-section, wherein the second complementary cross-section is an inverse shape complementary to the second cross-section, the distal portion adapted to engage with the distal region of the hollow body, and a medial transition portion extending between the proximal portion and the distal portion, the medial transition portion configured to mate with the medial transition region of the hollow body to form a passage, the passage in communication with the bore and adapted for routing the one or more flexible members around the shaft, and, the shaft including a tip extending distally from the distal portion of the shaft and beyond the distal region of the hollow body of the anchor;
displacing the one or more flexible members installed in the bore by inserting the shaft of the driver into the hollow body of the anchor until the one or more flexible members rests within the passage;
installing the anchor into a bone; and
tensioning the one or more flexible members.

11. The method of claim 10, further comprising threading one or more flexible members through the bore of the anchor.

12. The method of claim 11, further comprising positioning the one or more flexible members in one or more channels defined along at least a portion of an external surface of the hollow body of the anchor and extending along the longitudinal axis proximally from the bore.

13. The method of claim 10, wherein installing the anchor into the bone further comprises:
positioning the tip of the driver against a surface of the bone; and
applying an insertion force to the driver, wherein applying the insertion force causes the tip to penetrate the surface of the bone.

14. The method of claim 13, wherein installing the anchor into the bone further comprises continuing to apply the insertion force to the driver until the anchor is fully inserted into the bone.

15. The method of claim 13, wherein installing the anchor into the bone further comprises:
terminating application of the insertion force when the distal region of the anchor contacts the surface of the bone; and
screwing the anchor into the bone by twisting the driver until the anchor is fully inserted into the bone.

16. The method of claim 10, wherein the one or more flexible members include at least one of a wire, a suture, and suture tape.
